(19) United States
(12) Patent Application Publication
(10) Pub. No.: US 2012/0302496 A1
(43) Pub. Date: Nov. 29, 2012

(54) NOVEL 24-MEMBERED CYCLOOCTADEPSIPEPTIDES FROM FUNGAL STRAINS AND THEIR USE AS ANTHELMINTICS OR ENDOPARASITICIDES

(75) Inventors: Achim Harder, Koln (DE); Klemens Krieger, Lindlar (DE); Thi Lam Huong Pham, Berlin (DE); Thanh Dam Huynh, Berlin (DE); Irmtraut Zaspel, Muncheberg (DE); Dietrich Ewald, Golsdorf (DE); Clemens Mügge, Berlin (DE); Hardy Weisshoff, Berlin (DE)

(73) Assignee: BAYER INTELLECTUAL PROPERTY GMBH, MONHEIM (DE)

(21) Appl. No.: 13/514,538

(22) PCT Filed: Dec. 7, 2010
(86) PCT No.: PCT/EP2010/069040

§ 371 Date: Aug. 14, 2012

(30) Foreign Application Priority Data
Dec. 11, 2009 (EP) 0978799.4

(51) Int. Cl. C07K 11/02; A61P 33/10; A61P 33/00; C12P 21/04; A61K 38/15

(52) U.S. Cl. 514/4.6; 530/323; 435/71.1; 435/68.1

(57) ABSTRACT

The present invention relates firstly to the discovery of the previously unknown 24-membered cyclooctadepsipeptides PF1022-V, PF1022-W, XRB-C894, XRB-C942, XRB-C976, XRB-C1010, XRB-C1044, XRB-E922, XRB-E956, XRB-E990, XRB-E1024, XRB-S958, XRB-S992, XRB-S1026, XRB-S1060 (in various isomeric forms) and to the processes for the preparation of the abovementioned cyclooctadepsipeptides by means of the fungal strains from the family Xylariaceae, in particular the genera Rosellinia and Coniolariella, and by means of the mitosporic fungal strains from the groups Fusarium, Beauveria and Verticillium (orders Hypocreales and Phyllachorales), and by means of the enzymatic preparations isolated from these fungal strains, and secondly to the use of these strains for the preparation of the abovementioned novel cyclooctadepsipeptides and to the use of bassianolide and of the abovementioned novel cyclooctadepsipeptides individually or as a mixture, as anthelmintics or endoparasiticides.

The present invention relates firstly to the discovery of the novel 24-membered cyclooctadepsipeptides (the previously unknown derivatives PF1022-V and PF1022-W of the known anthelmintic cyclooctadepsipeptide PF1022-A and the previously unknown derivatives XRB-C894, XRB-C942, XRB-C976, XRB-C1010, XRB-C1044, XRB-E922, XRB-E956, XRB-E990, XRB-E1024, XRB-S958, XRB-S992, XRB-S1026, XRB-S1060 of the known cyclooctadepsipeptide bassianolide) and the processes for the preparation of the abovementioned cyclooctadepsipeptides by means of the fungal strains from the family Xylariaceae, in particular the genera Rosellinia and Coniolariella, and by means of the mitosporic fungal strains from the groups Fusarium, Beauveria and Verticillium (orders Hypocreales and Phyllachorales), and by means of the enzymatic preparations isolated from these fungal strains, and secondly the use of bassianolide and of the abovementioned cyclooctadepsipeptides, individually or as a mixture, as anthelmintics or endoparasiticides.

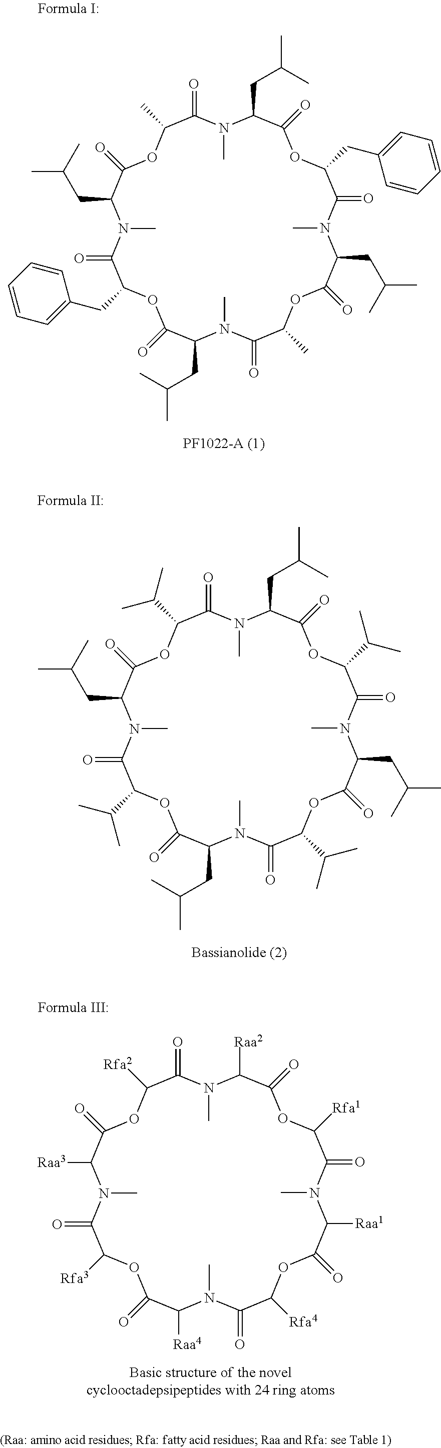

TABLE 1
Amino acid residues (Raa) and fatty acid residues (Rfa) of the novel 24-membered
cyclooctadepsipeptides (Nos. 3 to 17)

| Comp. No. | Name | Raa¹ | Rfa¹ | Raa² | Rfa² |
|---|---|---|---|---|---|
| 1 | PF1022-A | —CH₂CH(CH₃)₂ | —CH₃ | —CH₂CH(CH₃)₂ | —CH₂C₆H₅ |
| 2 | Bassianolide | —CH₂CH(CH₃)₂ | —CH(CH₃)₂ | —CH₂CH(CH₃)₂ | —CH(CH₃)₂ |
| 3 | PF1022-V⁽¹⁾ | —CH(CH₃)₂ | —CH₃ | —CH₂CH(CH₃)₂ | —CH₂C₆H₅ |
| 4 | PF1022-W | —CH₂CH(CH₃)₂ | —H | —CH₂CH(CH₃)₂ | —CH₂C₆H₅ |
| 5 | XRB-C894 | —CH(CH₃)₂ | —CH(CH₃)₂ | —CH₂CH(CH₃)₂ | —CH(CH₃)₂ |
| 6 | XRB-C942 | —CH₂C₆H₅ | —CH(CH₃)₂ | —CH₂CH(CH₃)₂ | —CH(CH₃)₂ |
| 7 | XRB-C976⁽¹⁾ | —CH₂C₆H₅ | —CH(CH₃)₂ | —CH₂CH(CH₃)₂ | —CH(CH₃)₂ |
| 8 | XRB-C1010⁽¹⁾ | —CH₂C₆H₅ | —CH(CH₃)₂ | —CH₂C₆H₅ | —CH(CH₃)₂ |
| 9 | XRB-C1044⁽²⁾ | —CH₂C₆H₅ | —CH(CH₃)₂ | —CH₂C₆H₅ | —CH(CH₃)₂ |
| 10 | XRB-E922 | —CH₂CH(CH₃)₂ | —C₄H₉ | —CH₂CH(CH₃)₂ | —CH(CH₃)₂ |
| 11 | XRB-E956⁽¹⁾ | —CH₂C₆H₅ | —C₄H₉ | —CH₂CH(CH₃)₂ | —CH(CH₃)₂ |
| 12 | XRB-E990⁽¹⁾ | —CH₂C₆H₅ | —C₄H₉ | —CH₂CH(CH₃)₂ | —CH(CH₃)₂ |
| 13 | XRB-E1024^{(1,2)} | —CH₂C₆H₅ | —C₄H₉ | —CH₂C₆H₅ | —CH(CH₃)₂ |
| 14 | XRB-S958 | —(CH₂)₂SO₂CH₃ | —CH(CH₃)₂ | —CH₂CH(CH₃)₂ | —CH(CH₃)₂ |
| 15 | XRB-S992⁽¹⁾ | —(CH₂)₂SO₂CH₃ | —CH(CH₃)₂ | —CH₂CH(CH₃)₂ | —CH(CH₃)₂ |
| 16 | XRB-S1026^{(1,2)} | —(CH₂)₂SO₂CH₃ | —CH(CH₃)₂ | —CH₂C₆H₅ | —CH(CH₃)₂ |
| 17 | XRB-S1060⁽²⁾ | —(CH₂)₂SO₂CH₃ | —CH(CH₃)₂ | —CH₂C₆H₅ | —CH(CH₃)₂ |

| Comp. No. | Raa³ | Rfa³ | Raa⁴ | Rfa⁴ |
|---|---|---|---|---|
| 1 | —CH₂CH(CH₃)₂ | —CH₃ | —CH₂CH(CH₃)₂ | —CH₂C₆H₅ |
| 2 | —CH₂CH(CH₃)₂ | —CH(CH₃)₂ | —CH₂CH(CH₃)₂ | —CH(CH₃)₂ |
| 3 | —CH₂CH(CH₃)₂ | —CH₃ | —CH₂CH(CH₃)₂ | —CH₂C₆H₅ |
| 4 | —CH₂CH(CH₃)₂ | —CH₃ | —CH₂CH(CH₃)₂ | —CH₂C₆H₅ |
| 5 | —CH₂CH(CH₃)₂ | —CH(CH₃)₂ | —CH₂CH(CH₃)₂ | —CH(CH₃)₂ |
| 6 | —CH₂CH(CH₃)₂ | —CH(CH₃)₂ | —CH₂CH(CH₃)₂ | —CH(CH₃)₂ |
| 7 | —CH₂C₆H₅ | —CH(CH₃)₂ | —CH₂CH(CH₃)₂ | —CH(CH₃)₂ |
| 8 | —CH₂C₆H₅ | —CH(CH₃)₂ | —CH₂CH(CH₃)₂ | —CH(CH₃)₂ |
| 9 | —CH₂C₆H₅ | —CH(CH₃)₂ | —CH₂C₆H₅ | —CH(CH₃)₂ |
| 10 | —CH₂CH(CH₃)₂ | —CH(CH₃)₂ | —CH₂CH(CH₃)₂ | —CH(CH₃)₂ |
| 11 | —CH₂CH(CH₃)₂ | —CH(CH₃)₂ | —CH₂CH(CH₃)₂ | —CH(CH₃)₂ |
| 12 | —CH₂C₆H₅ | —CH(CH₃)₂ | —CH₂CH(CH₃)₂ | —CH(CH₃)₂ |
| 13 | —CH₂C₆H₅ | —CH(CH₃)₂ | —CH₂CH(CH₃)₂ | —CH(CH₃)₂ |
| 14 | —CH₂CH(CH₃)₂ | —CH(CH₃)₂ | —CH₂CH(CH₃)₂ | —CH(CH₃)₂ |
| 15 | —CH₂C₆H₅ | —CH(CH₃)₂ | —CH₂CH(CH₃)₂ | —CH(CH₃)₂ |
| 16 | —CH₂C₆H₅ | —CH(CH₃)₂ | —CH₂CH(CH₃)₂ | —CH(CH₃)₂ |
| 17 | —CH₂C₆H₅ | —CH(CH₃)₂ | —CH₂C₆H₅ | —CH(CH₃)₂ |

⁽¹⁾including various structural isomers
⁽²⁾occurs at very low concentrations
PF1022A (see formula I) is outstandingly suitable for controlling endoparasites, especially in the field of human and veterinary medicine. Such cyclooctadepsipeptides with 24 ring atoms and their use as endoparasiticides are already the subject matter of an earlier patent application (US005571793A).

Bassianolide (see formula II) has become known as an antibiotic with insecticidal activity (Kanaoka, M. et al., Bassianolide, a New Insecticidal Cyclodepsipeptide from Beauveria bassiana and Verticillium lecanii; Agric. Biol. Chem. 42 (1978), 629-635). A series of chemical and microbial processes exists for the preparation of cyclic, D-2-hydroxy-isovaleric acid-containing depsipeptides with 24 ring atoms (for example by means of synthesis, cf.: Ohyama M. et al., Biosci. Biotechnol. Biochem. 58 (1994) p. 1193-1194; Scherkenbeck J. et al. Tetrahedron 51 (1995) p. 8459-8470; Kobayashi M. et al. Annu. Rep. Sankyo Res. Lab. 46 (1994) p. 67-75; Lee B. et al. Bioorg. Med. Chem. Lett. 12 (2002) p. 353-356; Dutton F E et al. J. Antibiot. 47 (1994) p. 1322-1327).

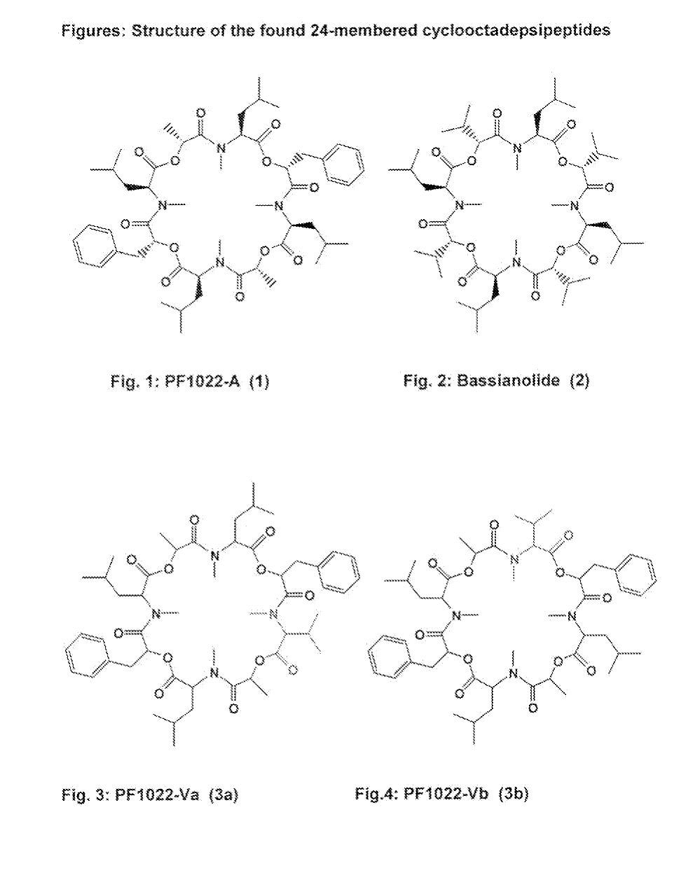

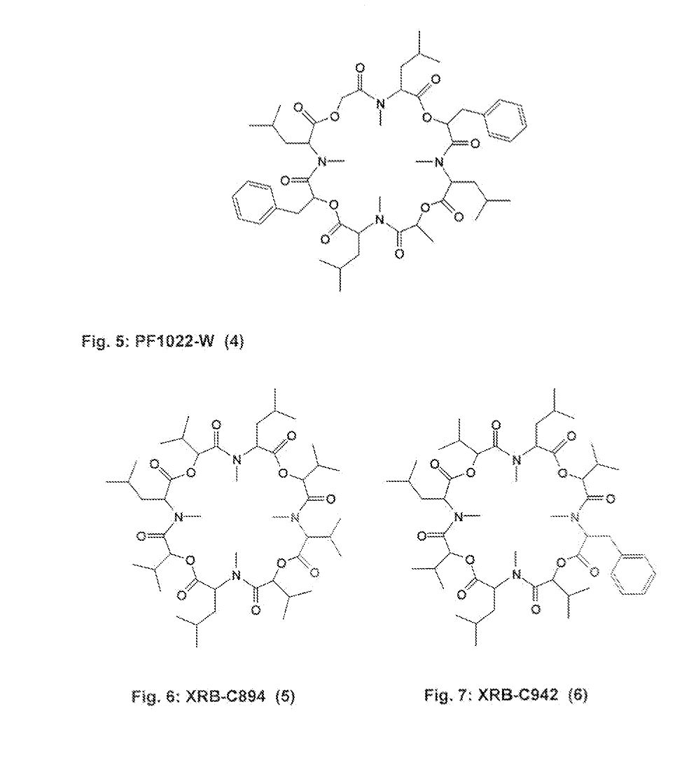

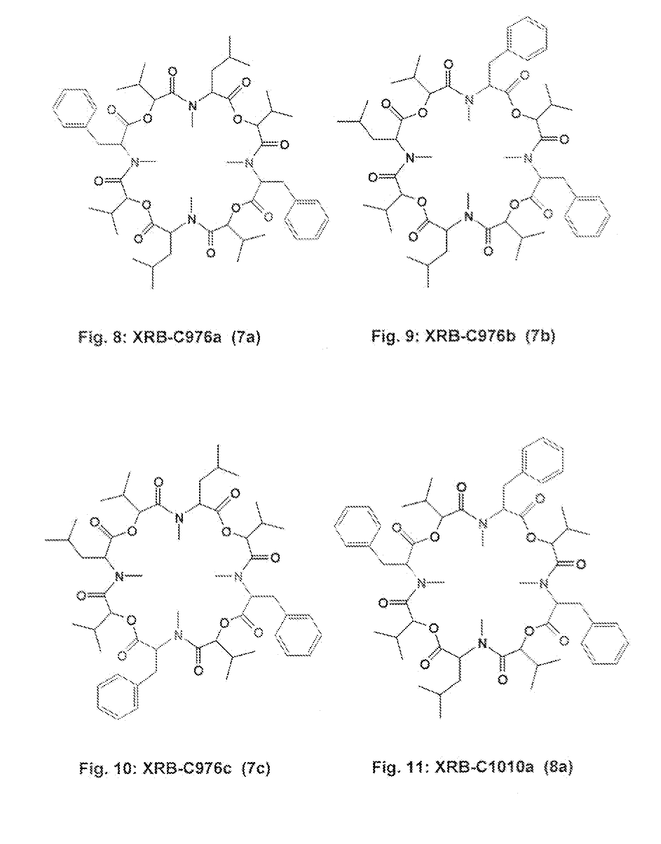

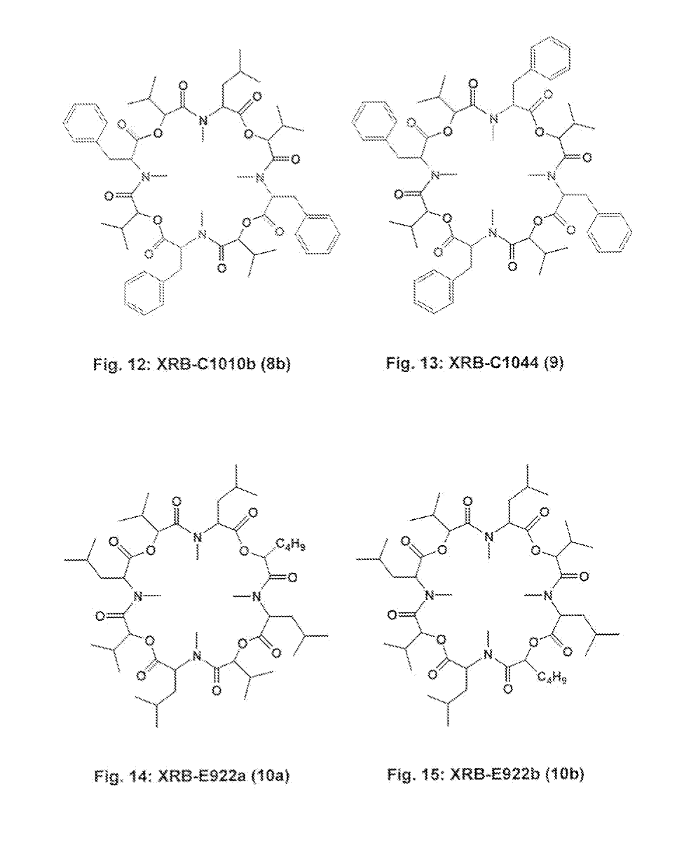

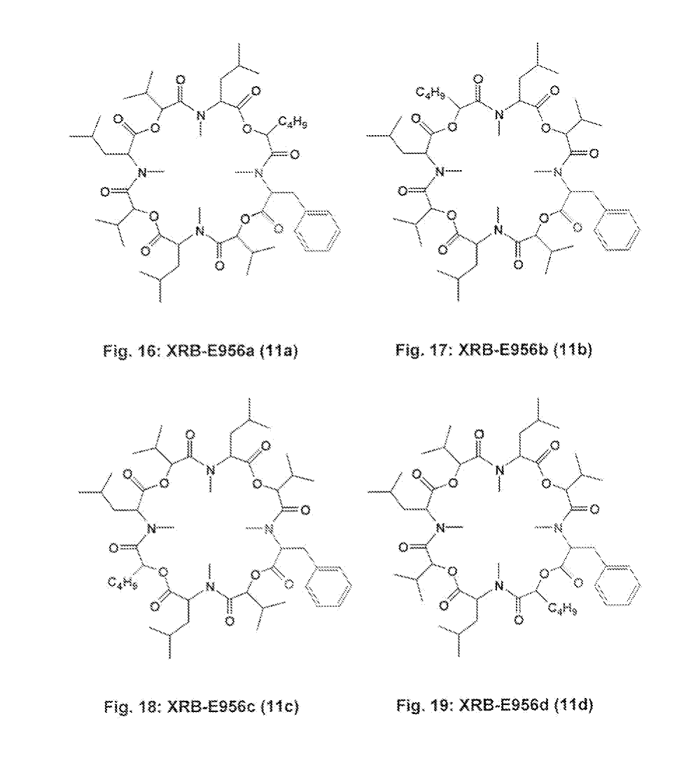

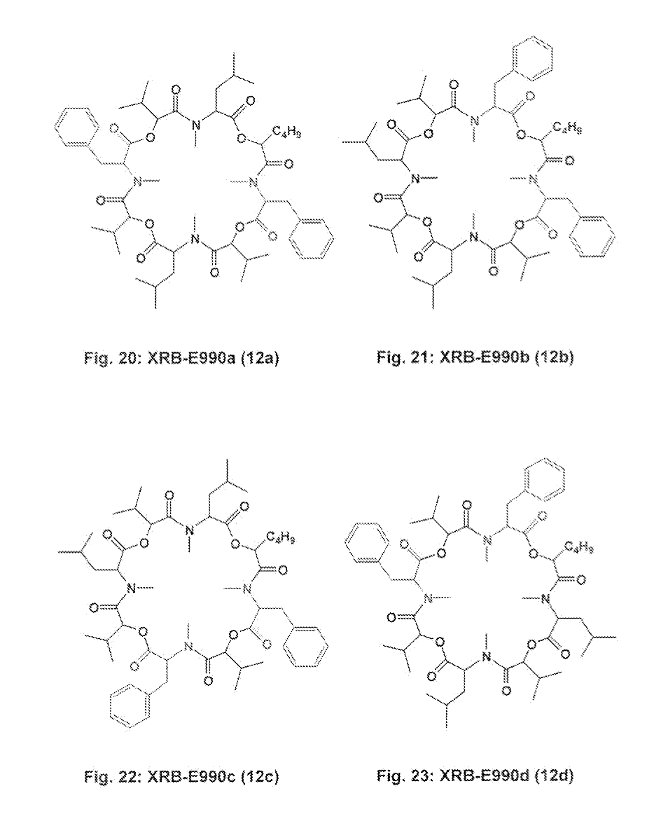

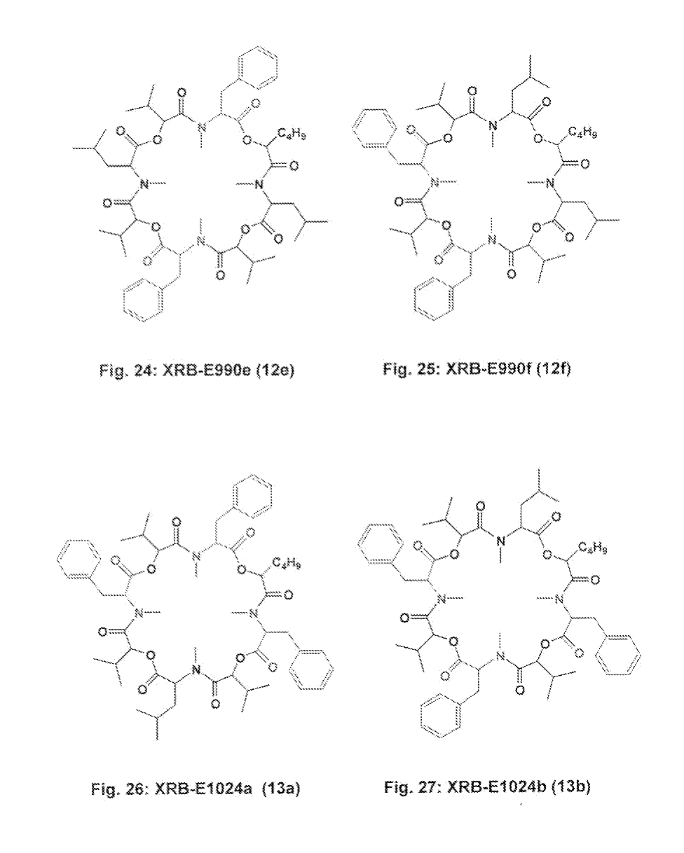

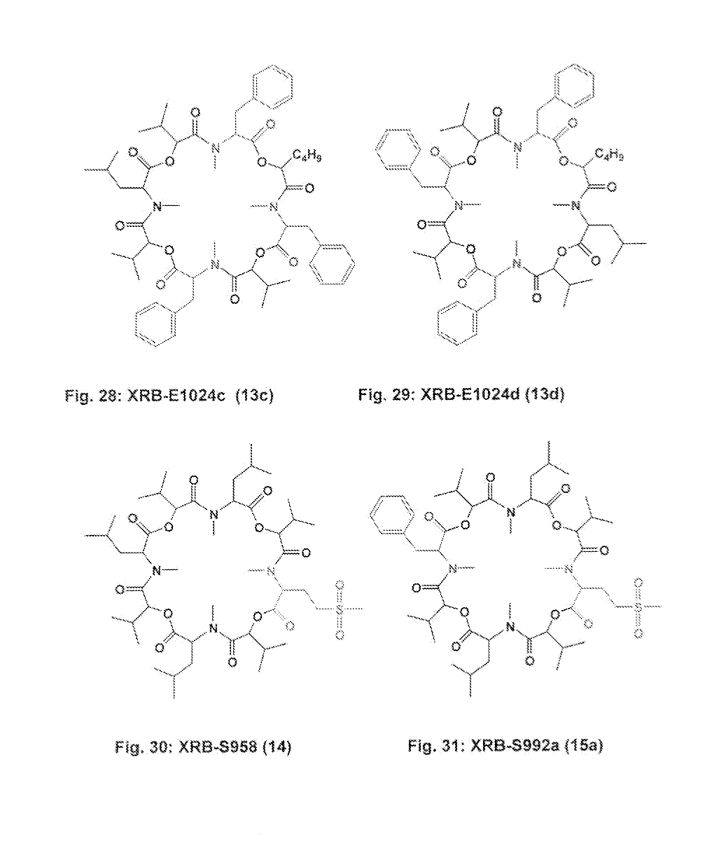

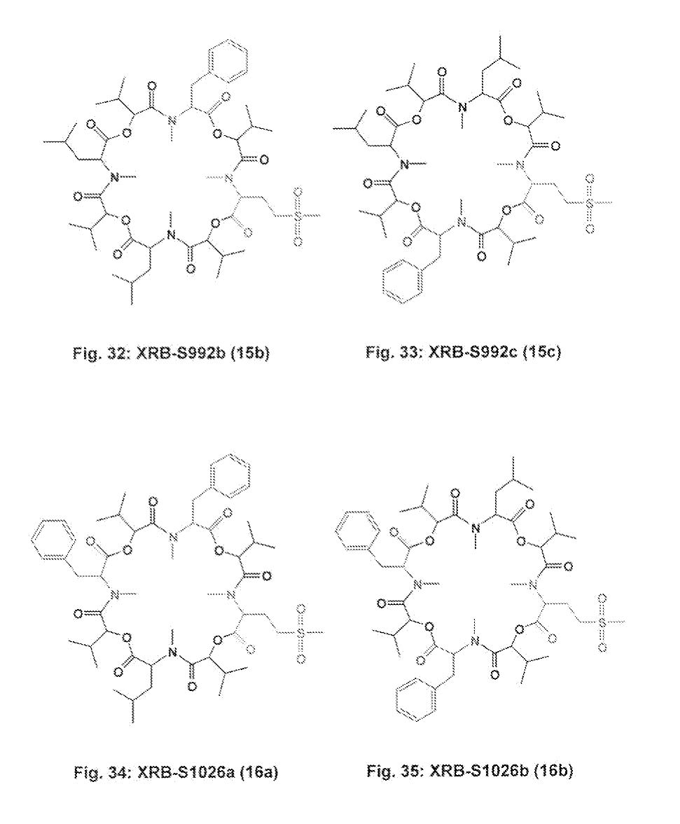

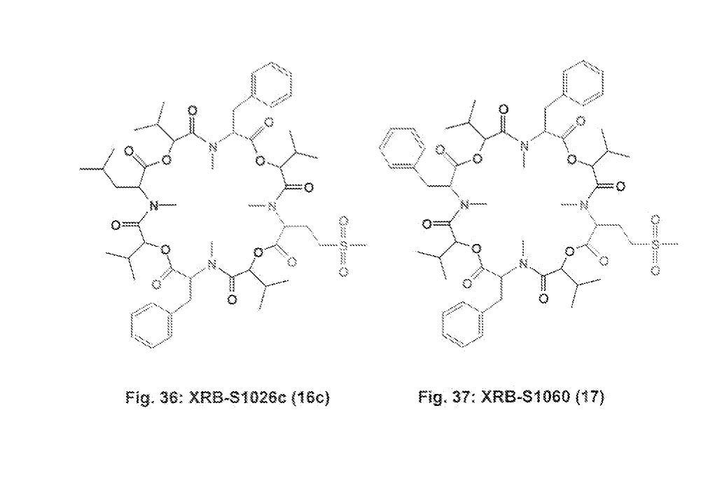

The present invention relates to the discovery of the previously unknown 24-membered cyclooctadepsipeptides (PF1022-V, PF1022-W, XRB-C894, XRB-C942, XRB-C976, XRB-C1010, XRB-C1044, XRB-E922, XRB-E956, XRB-E990, XRB-E1024, XRB-S958, XRB-S992, XRB-S1026, XRB-S1060) in various isomeric forms (see formula III, Table 1 and FIGS. 3 to 37) and to the processes for the preparation of the abovementioned cyclooctadepsipeptides from the fungal strains from the family Xylariaceae, in particular the genera Rosellinia and Coniolariella, which are obtained from fruiting bodies of these representatives and further Xylariaceae in dead timber of broad-leaved trees and coniferous trees or from purchased, known strains of strain collections by multiple selections, and from the mitosporic fungal strains from the groups Fusarium, Beauveria and Verticillium (orders Hypocreales and Phyllachorales), and by means of the enzymatic preparations isolated from these abovementioned fungal strains.

### Description of the Generation or Isolation of Fungal Strains or Fungal Lines as Effective Producers of Novel 24-Membered Cyclooctadepsipeptides:

The present invention relates to the preparation of novel 24-membered cyclooctadepsipeptides (PF1022-V, PF1022-W, XRB-C894, XRB-C942, XRB-C976, XRB-C1010, XRB-C1044, XRB-E922, XRB-E956, XRB-E990, XRB-E1024, XRB-S958, XRB-S992, XRB-S1026, XRB-S1060) in various isomeric forms by means of the fungal strains from the family Xylariaceae, in particular the genera Rosellinia and Coniolariella, within which several fungal lines are capable of producing the cyclooctadepsipeptides.

Fungal strains from the family Xylariaceae, in particular the genus Rosellinia, were purchased from various strain collections in order to find novel cyclooctadepsipeptide producers. In order to obtain suitable fungal lines as efficient cyclooctadepsipeptide producers, the abovementioned fungal strains were improved in respect of the cyclooctadepsipeptide production by means of multiple selections. Furthermore, new isolates were obtained from fruiting bodies of Rosellinia and Coniolariella and from other Xylariaceae in the dead timber of various broad-leaved and coniferous tree species, and novel lines were obtained from these new isolates after selections in respect of their cyclooctadepsipeptide production. A variety of mitosporic fungi from the orders Hypocreales and Phyllachorales, in particular the groups Fusarium, Beauveria and Verticillium, may occur as accompanying organisms, and these are likewise capable of producing cyclooctadepsipeptides.

The fungal strains Rosellinia abscondita (CBS 447.89, CBS 448.89, CBS 450.89), R. britannica (CBS 446.89), R. mammaeformis (CBS 445.89), R. millegrana (CBS 111.75), R. nectrioides (CBS 449.89), R. necatrix (CBS 267.30), Xylaria hypoxylon (CBS 499.80) were deposited in 1930, 1975 or 1989 (according to the CBS code) at the strain collection of the CBS (Centraalbureau voor Schimmelcultures, Institute of the Royal Netherlands Academy of Arts and Sciences, the Netherlands) and obtained therefrom in the form of cultures. The lines XR-909, XR-912, XR-913, XR-916 and XR-917, which differ in culture from the original strains by their growth habit and their capability for producing cyclooctadepsipeptides, were obtained from these CBS fungal strains after repeated selections on mSeed medium. The property of mycelial development is linked to the production of certain cyclooctadepsipeptides. Other fungal strains were isolated from natural sources.

The genus Rosellinia is a ubiquitous fungal genus of the family Xylariaceae, which belongs to the extensive order Xylariales (phylum Ascomycota).

The representatives of the genus Rosellinia are found on the bark of broad-leaved and coniferous trees in coolish locations of the moderate zones of the northern hemisphere. They live predominantly saprophytically on old or dead timber which is already in the process of degradation, or on herbaceous plants. They have characteristic spherical hard fruiting bodies, so-called perithecia with ostioles, in which asci with the species-typical spores develop. The asci are characterized by species-typical cubical or rectangular apical apparatuses which serve for ejecting the mature spores. The spores of the genus Rosellinia have species-typical germination slits.

The spores of R. abscondita are pale brown and between 16 to 24×5.5 to 8.5 μm in size. Very frequently, the germination slit is arranged diagonally over the spore surface. The spore poles bear a mucous sheath. The spores of R. britannica are 22 to 27×7 to 9 μm in size, brown and bear mucous sheaths. The spores of R. mammaeformis are 17 to 21×6 to 7 μm in size, brown with darker ends. The species R. nectrioides is very similar to R. abscondita. The asco spores are likewise pale brown, but there are no appendages or mucous sheaths.

The perithecia of the species are usually embedded in a dark brown to black subiculum (thick layers of hyphae). The hyphae grow from the subiculum into the bark tissue and the layers therebelow. The individual species make high demands on the environmental conditions which occur under natural conditions. Mature perithecia occur in mild winters and in the early months of the year. In general, the abovementioned Rosellinia species are rare.

The genus Coniolariella was renamed after various species, which had previously been classified under Rosellinia, had been classified as belonging to this genus (Checa et al. (2008) Mycol. Res. 112, 7, 795-801).

Obtaining Pure Cultures from Natural Habitats: Pure cultures (from fruiting bodies of Rosellinia, Coniiolariella and further Xylariaceaen in dead timber of broad-leaved and coniferous trees) were obtained from mature, intact perithecia which were as isolated as possible and which had been removed from dead timber. They were surface-disinfected with 0.05% strength AgNO₃ solution and rinsed repeatedly with sterile water. The perithecia were carefully crushed on a glass slide, and the asci and spores which were released were transferred into tubes containing sterile water.

By viewing under the microscope, it was possible to estimate the density and maturity stage at which the spores occurred. If the spore density was high, the spore suspension was diluted by a factor of ten, and 100 μl of the respective dilution stage were plated onto malt extract agar (MEA). The plates were stored at 18 to 20° C., scored every day under the microscope, and germinating spores were transferred to fresh potato dextrose agar (PDA). The mycelium formed which grows on this medium is whitish, flat and delicate; no aerial mycelium develops.

Besides the representatives of Xylariaceae, various mitosporic fungi which were classified as belonging, inter alia, to the groups Fusarium, Verticillium and Beauveria, grow at different densities. Pure cultures of these forms were likewise established.

After the cultures had grown sufficiently (colony sizes of about 85 mm), the mycelium was extracted with methanol. The constituents of the mycelial extracts were identified by means of LC-PDA-ESI-Q-TOF-MS and -MS/MS. Various lines of Rosellinia and Coniolariella, which proved to be positive in respect of the production of 24-membered cyclooctadepsipeptides, in particular of PF1022-A (see patent BHC 08 8 004, 16 Dec. 2008) and of its derivatives PF1022-V and PF1022-W, were found. These include, for example, strain XR-21 (Rosellinia corticum from broad-leaved wood).

Since the present cultures are isolates which have been transferred directly from the natural ecosystem to artificial conditions, it is possible that culture forms appear which develop different mycelial forms and a variation in the intensity of the production of active substance. This is why the isolates obtained were improved by multiple selections in order to find cyclooctadepsipeptide production strains.

The fungal lines were selected for further propagation on the basis of the cyclooctadepsipeptide content, the cell growth and the mycelial form. Multiple selections and passages gave rise to fungal lines with a typical mycelial form, more rapid growth and better cyclooctadepsipeptide production capacity.

Assaying the Growth of Fungal Lines:

The growth of the fungal lines of the Xylariaceae in culture on malt extract agar (MEA), potato dextrose agar (PDA), cornmeal agar (CMA) or mSeed agar tends to be slow and to a high degree temperature-dependent. They develop a whitish flat delicate mycelium, in some cases also a filamentous mycelium, on these media.

In contrast to the original cultures, only a small amount of aerial mycelium is developed. It is only in older cultures that a white fine felty surface is formed, while a dark leather-like layer develops in agar. Line XR-909 is an exception in as far as the colour of the mycelium changes relatively rapidly to dark greyish-brown, even at the surface.

Line XR-916 shows the highest growth rate at 15° C. and, after 15 days, reaches a colony size of 85 mm on MEA and a colony size of 70 mm on PDA. Under these conditions, the growth of line XR-909 on MEA amounts to 62 mm and on PDA to 52 mm. Line XR-913 shows a growth of 85 mm diameter on PDA and of 68 mm diameter on MEA. Line XR-917 shows the same growth on both media and reaches colony sizes of in each case 85 mm.

At 21° C., the growth of the newly obtained lines is poorer. After 15 days, line XR-916 reaches 46 mm on PDA and 51 mm on MEA. During this time, line XR-909 reaches 45 mm on PDA and 50 mm on MEA. Other lines have colony sizes of 85 mm on both media at 21° C.

At 27° C., the newly obtained lines have ceased to grow, only line XR-913 reaches, after 15 days, a diameter of 52 mm on PDA and of 42 mm on MEA.

Some of the lines, such as XR-916, develop dark vesicles in the agar, which can be discerned under the microscope. The development of spores or conidia of the corresponding imperfect stage of the Xylariaceae in culture is observed very rarely.

Fusarium and Verticillium isolates show good growth at temperatures between 22 and 25° C. and Beauveria isolates at 20° C. (on mSeed medium).

Composition of the media used:

MEA: malt extract 17 g/l, agar 15 g/l

PDA: glucose 5 g/l, potato starch 20 g/l, agar 15 g/l

CMA: cornmeal 50 g/l, agar 15 g/1

mSeed agar: Pharmamedia 20 g/l, soya peptone 2 g/l, maltose 40 g/l,

### Description of the Isolation and Identification of Novel 24-Membered Cyclooctadepsipeptides:

To rapidly identify the constituents, the methanolic mycelial extracts from the CBS fungal strains (CBS 111.75, CBS 267.30, CBS 445.89, CBS 446.89, CBS 447.89, CBS 448.89, CBS 449.89, CBS 450.89, CBS 499.80) and from the newly obtained lines or isolates (XR-909, XR-913, XR-916, XR-917, XR-21, etc.), and also from further fungal strains from the groups Fusarium, Beauveria and Verticillium were first analysed by means of LC-PDA-ESI-Q-TOF-MS.

The detailed studies on the identification of the constituents were performed by means of LC-ESI-Q-TOF-MS/MS and -pseudoMS³ in aqueous and in deuterated solvents (H/D exchange experiments). Many known, but also novel, cyclic tetra-, hexa-, octa- and deca-depsipeptides in the mycelial extracts of the abovementioned fungal strains were identified by means of the abovementioned MS techniques.

Although the abovementioned CBS fungal strains have already been deposited in the CBS strain collection between 1930 and 1989, no published data are available as yet on the discovery of the cyclodepsipeptides from these strains. This is the first time that bassianolide from Rosellinia strains have been found.

To isolate the novel 24-membered cyclooctadepsipeptides for biological tests, the cell material was obtained from agar plates and extracted with methanol. The methanolic extracts were concentrated in vacuo until free from methanol, and then extracted repeatedly with ethyl acetate. The ethyl acetate extracts were concentrated in vacuo until free from ethyl acetate, then dried by lyophilization until free from water and subsequently separated by means of column chromatography (Silica Gel 60, n-hexane/ethyl acetate/methanol) into coarse fractions. Cyclooctadepsipeptides are obtained from the coarse fractions by means of preparative HPLC separation.

The detailed structure elucidation for novel main cyclooctadepsipeptides is performed by means of 1D- and 2D-NMR spectroscopy (¹H, ¹³C, DEPT, HMQC, HMBC, COSY, NOESY . . . ).

From the abovementioned CBS fungal strains and from the newly obtained lines or isolates (XR-21, XR-909, XR-913, XR-916, XR-917), the novel 24-membered cyclooctadepsipeptides (PF1022-V, PF1022-W, XRB-C894, XRB-C942, XRB-C976, XRB-C1010, XRB-C1044, XRB-E922, XRB-E956, XRB-E990, XRB-E1024, XRB-S958, XRB-S992, XRB-S1026, XRB-S1060) were identified, partially isolated and tested (both as a mixture and individually).

Owing to the positive results of the in-vitro tests with Trichinella and Nippostrongylus, the use of the abovementioned novel 24-membered cyclooctadepsipeptides (in various isomeric forms), individually or as a mixture, is herewith patented as anthelmintics or endoparasiticides.

### Analytical Data:

### 1. Working Condition for the Isolation and Identification

1.1. Coarse Separation of the Extract of Strain XR-916 by Means of Column chromatography:

Sample size: 5 g of dried ethyl acetate extract

Separating material: 30 g Silica Gel 60 (15-40 μm, Merck)

Column dimensions: 20 cm×2 cm

Flow rate: 2 ml/min

Fraction volume: 50 ml

### Mobile Phase:

Fraction 1-2: n-hexane/ethyl acetate: 2/1

Fraction 3-4: n-hexane/ethyl acetate: 1/1

Fraction 5-6: n-hexane/ethyl acetate: 1/2

Fraction 7-8: n-hexane/ethyl acetate: 1/3

Fraction 9-10: n-hexane/ethyl acetate: 1/4

Fraction 11-12: n-hexane/ethyl acetate: 1/9

Fraction 13-14: ethyl acetate

Fraction 15-16: ethyl acetate/methanol: 98/2

Fraction 17-18: ethyl acetate/methanol: 95/5

Fraction 19-20: ethyl acetate/methanol: 90/10

Fraction 21-22: ethyl acetate/methanol: 85/15

Fraction 23-26: ethyl acetate/methanol: 80/20

### Constituents of the Fractions:

Fraction 2-4: bassianolide, XRB-C894, -C942 and -C976,
XRB-D- and -E cyclooctadepsipeptides, XRB-F cyclodecadepsipeptides

Fraction 5-7: XRB-C942, XRB-C976, XRB-B cyclohexadepsipeptides

Fraction 8-16: XRB-C976, -C1010 and -C1044, XRB-A linear peptides, XRB-B peptides

Fraction 17-25: enniatin C, XRB-S958, XRB-S992, XRB-S1026, XRB-S1060

### 1.2. Group Separation of the Coarse Fractions of Strain XR-916 by Means of HPLC:

HPLC column: Luna C5 (5 μm, 250×4.6 mm)

Eluent A: water (0.1% HCOOH)

Eluent B: methanol (0.1% HCOOH)

Eluent C: acetonitrile (0.1% HCOOH)

HPLC condition:

| Time | A | B | C | Curve | Flow rate |
|---|---|---|---|---|---|
| 0 min | 25 | 65 | 10 | 1 | 1 ml/min |
| 33 min | 20 | 65 | 15 | 6 | 1 ml/min |
| 34 min | 5 | 65 | 30 | 1 | 1 ml/min |
| 38 min | 5 | 65 | 30 | 1 | 1 ml/min |
| 39 min | 25 | 65 | 10 | 1 | 1 ml/min |
| 45 min | 25 | 65 | 10 | 1 | 1 ml/min |

Column temperature: 40° C.

Injection volume: 15 μl

PDA detector: 210 to 400 nm

Fraction: 20 sec/fr.×120 fr. (wait time: 1.75 min)

Results:

Fraction with Rₜ 10 to 20 min: cyclo- and linear depsipeptides of the XRB-S, -A, -B group

Fraction with Rₜ 20 to 35 min: cyclodepsipeptides of the XRB-C, -D, -E and -F group

### 1.3. Fine Separation of the Depsipeptides of Strain XR-916 by Means of HPLC

1.3.1. Separation Condition for Polar Depsipeptides (Fr. with Rₜ from 10 to 20 Min):

HPLC column: Luna C5 (5 μm, 250×4.6 mm)

Eluent A: water (0.1% HCOOH)

Eluent B: acetonitrile (0.1% HCOOH)

HPLC condition: isocratic (A/B: 33/67)

Running time: 45 min

Flow rate: 0.6 ml/min

Column temperature: 40° C.

Injection volume: 10 μl

PDA detector: 210 to 400 nm

Fractions: 18 sec/fr.×120 fr. (wait time: 15 min)

Rₜ from 19.8 to 20.5 min: linear depsipeptide XRB-A767
(C₄₂H₆₁N₃O₁₀, [M+H]⁺ m/z 768.4435)

Rₜ from 21.3 to 22.5 min: linear depsipeptide XRB-A733
(C₃₉H₆₃N₃O₁₀, [M+H]⁺ m/z 734.4592)

Rₜ from 23.1 to 24.9 min: linear depsipeptide XRB-A699
(C₃₆H₆₅N₃O₁₀, [M+H]⁺ m/z 700.4748)

Rₜ from 31.8 to 32.7 min: XRB-S992

Rₜ from 33.3 to 35.4 min: XRB-S958

Rₜ from 37.8 to 40.2 min: enniatin C (C₃₆H₆₃N₃O₉, [M+H]⁺ m/z 682.4643)

1.3.2. Separation Condition for Cyclooctadepsipeptides (Fr. with Rₜ from 20 to 35 min):

HPLC column: Luna C5 (5 μm, 250×4.6 mm)

Eluent A: water (0.1% HCOOH)

Eluent B: acetonitrile (0.1% HCOOH)

HPLC condition: isocratic (A/B: 25/75)

Running time: 25 min

Flow: 1 ml/min

Column temperature: 40° C.

Injection volume: 10 μl

PDA detector: 210 to 400 nm

Fractions: 18 sec/fr.×120 fr. (wait time: 2 min) Rₜ 15.8 to 16.5 min: XRB-C1044

Rₜ 17.3 to 18.3 min: XRB-C1010 Rₜ 19.3 to 20.8 min: XRB-C976

Rₜ 22 to 23.5 min: XRB-C942

### 1.4. Conditions of the LC-MS, -MS/MS and -MS-H/D Exchange Experiments

### 1.4.1. Conditions of the LC-MS and -MS/MS Experiments for Strain XR-21

HPLC column: GeminiNX C18 (3 μm, 150×2 mm)

Eluent A: water/acetonitrile: 9/1 (0.1% HCOOH)

Eluent B: acetonitrile (0.1% HCOOH)

HPLC condition:

| Time | A | B | Curve | Flow rate |
|---|---|---|---|---|
| 0 min | 40 | 60 | 1 | 0.25 ml/min |
| 36 min | 20 | 80 | 6 | 0.25 ml/min |
| 37 min | 5 | 95 | 1 | 0.35 ml/min |
| 42 min | 5 | 95 | 1 | 0.35 ml/min |
| 43 min | 40 | 60 | 1 | 0.25 ml/min |
| 50 min | 40 | 60 | 1 | 0.25 ml/min |

Column temperature: 40° C.

Injection volume: 5 μl

PDA detector: 210 to 400 nm

MS detector: ESI-Q-TOF-MS (full-scan, m/z 130 to 2000)

### 1.4.2. Conditions of the LC-MS and -MS/MS Experiments for Strain XR-916:

HPLC column: GeminiNX C18 (3 μm, 150×2 mm)
18

Eluent A: water/acetonitrile: 9/1 (0.1% HCOOH)

Eluent B: acetonitrile (0.1% HCOOH)

HPLC condition:

| Time | A | B | Curve | Flow rate |
|---|---|---|---|---|
| 0 min | 40 | 60 | 1 | 0.2 ml/min |
| 105 min | 38 | 62 | 6 | 0.2 ml/min |
| 108 min | 25 | 75 | 6 | 0.35 ml/min |
| 140 min | 5 | 95 | 1 | 0.35 ml/min |
| 143 min | 5 | 95 | 1 | 0.35 ml/min |
| 144 min | 40 | 60 | 1 | 0.35 ml/min |
| 150 min | 40 | 60 | 1 | 0.35 ml/min |

Column temperature: 40° C.

Injection volume: 10 μl

PDA detector: 210 to 400 nm

MS detector: ESI-Q-TOF-MS (full-scan, m/z 130 to 2000)

### 1.4.3. Conditions of the LC-MS-H/D Exchange Experiments:

HPLC column: GeminiNX C18 (3 μm, 150×2 mm)
18

Eluent A: D₂O (0.1% DCOOD)

Eluent B: acetonitrile (0.1% DCOOD)

HPLC condition:

| Time | A | B | Curve | Flow rate |
|---|---|---|---|---|
| 0 min | 35 | 65 | 1 | 0.2 ml/min |
| 80 min | 32 | 68 | 6 | 0.2 ml/min |
| 87 min | 5 | 95 | 1 | 0.2 ml/min |
| 92 min | 5 | 95 | 1 | 0.2 ml/min |
| 93 min | 35 | 65 | 1 | 0.2 ml/min |
| 100 min | 35 | 65 | 1 | 0.2 ml/min |

Column temperature: 40° C.

Injection volume: 10 μl

PDA detector: 210 to 400 nm

MS detector: ESI-Q-TOF-MS (full-scan, m/z 130 to 1600)

Analytical Data of the Cyclooctadepsipeptides with 24 Ring Atoms

### 2.1. Analytical Data of PF1022-A (1), PF1022-V (3) and PF1022-W (4)

### 2.1.1. PF1022-A (1)

Empirical formula: C₅₂H₇₆N₄O₁₂

Accurate mass: 948.5460

[M+H]⁺ (m/z): found: 949.5540; calculated: 949.5538

ESI-MS/MS of m/z 949.6 (Rₜ=23.6 min, collision energy 25, 40, 75 V) [m/z (rel. int.)]: 100.1 (45), 140.1 (10), 172.1 (25), 182.1 (72), 200.1 (55), 216.1 (5), 230.1 (<5), 248.1 (23), 258.1 (100), 276.1 (55), 348.2 (18), 475.3 (19), 493.3 (5), 547.3 (<5), 595.4 (<5), 620.3 (<5), 674.4 (5), 750.4 (5), 768.5 (<5), 822.5 (<5), 949.6 (15).

### 2.1.2. PF1022-V (3)

Empirical formula: C₅₁H₇₄N₄O₁₂

Accurate mass: 934.5303

[M+H]⁺ (m/z): found: 935.5390; calculated: 935.5382

ESI-MS/MS of m/z 935.5 (Rₜ=23.4 min, collision energy: 25, 40, 75 V) [m/z (rel. int.)]: 86.1 (48), 100.1 (80), 140.1 (18), 154.1 (10), 172.1 (40), 182.1 (68), 200.1 (75), 218.1 (10), 230.1 (6), 248.1 (22), 258.1 (100), 276.1 (51), 285.1 (30), 313.2 (30), 348.2 (22), 388.2 (15), 445.3 (12), 460.3 (15), 475.3 (20), 587.4 (18), 662.4 (15), 738.4 (22), 919.5 (55), 937.5 (70).

### 2.1.3. PF1022-W (4)

Empirical formula: C₅₁H₇₄N₄O₁₂

Accurate mass: 934.5303

[M+H]⁺ (m/z): found: 935.5390; calculated: 935.5382

ESI-MS/MS of m/z 935.5 (Rₜ=24.4 min, collision energy: 25, 40, 75 V) [m/z (rel. int.)]: 100.1 (45), 112.1 (11), 140.1 (22), 172.1 (28), 182.1 (65), 200.1 (100), 218.1 (10), 248.1 (12), 258.1 (55), 276.1 (28), 334.2 (12), 348.2 (12), 434.3 (11), 461.3 (22), 475.3 (12), 533.4 (12), 551.4 (12), 660.4 (12), 708.5 (22), 935.5 (20).

ESI-MS/MS of m/z 935.5 (Rₜ=26 min, collision energy. 25, 40, 75 V) [m/z (rel. int.)]: 100.1 (40), 140.1 (5), 158.1 (8), 172.1 (15), 182.1 (40), 200.1 (22), 216.1 (10), 248.1 (28), 258.1 (100), 276.1 (60), 348.2 (20), 433.3 (9), 461.3 (10), 475.3 (12), 533.4 (6), 632.4 (8), 660.4 (8), 709.4 (5), 736.4 (6), 760.4 (5), 808.4 (8), 907.5 (6), 935.5 (8).

2.2. Analytical Data of Bassianolide and of its Novel Derivatives (XRB-C894, XRB-C942, XRB-C976, XRB-C1010, XRB-C1044, XRB-D940, XRB-D974, XRB-E922, XRB-E956, XRB-E990, XRB-E1024, XRB-S958, XRB-S992, XRB-S1026, XRB-S1060) Including their Isomers

### 2.2.1. Bassianolide (2)

Empirical formula: C₄₈H₈₄N₄O₁₂

Accurate mass: 908.6086 [M+H]⁺ (m/z): found: 909.6166; calculated: 909.6164

H/D exchange experiment: [M+D]⁺ m/z 910.63, [M+Na]⁺ m/z 931.6 (Rₜ=49.7 min)

ESI-MS/MS of m/z 909.6 (Rₜ=70 min, collision energy: 25 and 85 V) [m/z (rel. int.)]: 100.1 (55), 126.1 (15), 168.1 (11), 200.1 (13), 210.1 (100), 228.1 (65), 328.2 (8), 455.3 (18), 473.3 (8), 555.4 (5), 600.4 (5), 654.4 (<5), 682.4 (10), 700.4 (7), 782.4 (5), 909.6 (21).

### 2.2.2. XRB-C894 (5)

Empirical formula: C₄₇H₈₂N₄O₁₂

Accurate mass: 894.5929

[M+H]⁺ (m/z): found: 895.5987; calculated: 895.6008

ESI-MS/MS of m/z 895.6 (Rₜ=79.8 min, collision energy. 25 and 85 V) [m/z (rel. int.)]: 86.1 (5), 100.1 (48), 126.1 (8), 168.1 (5), 200.1 (8), 210.1 (100), 228.1 (38), 313.2 (<5), 328.2 (8), 413.3 (5), 441.3 (8), 455.3 (7), 541.3 (<5), 555.4 (<5), 640.4 (6), 668.4 (6), 686.4 (6), 768.4 (<5), 895.5 (10).

### 2.2.3. XRB-C942 (6)

Empirical formula: C₅₁H₈₂N₄O₁₂

Accurate mass: 942.5929

[M+H]⁺ (m/z): found: 943.6023; calculated: 943.6008

H/D exchange experiment: [M+D]⁺ m/z 944.61, [M+Na]⁺ m/z 965.59 (Rₜ=47.5 min)

ESI-MS/MS of m/z 943.6 (Rₜ=67 min, collision energy: 25 and 85 V) [m/z (rel. int.)]: 100.1 (52), 126.1 (10), 134.1 (22), 168.1 (5), 200.1 (10), 210.1 (100), 228.1 (68), 244.1 (21), 262.1 (20), 328.2 (8), 455.3 (13), 473.3 (5), 489.3 (11), 507.3 (5), 555.4 (<5), 589.4 (<5), 634.4 (<5), 682.4 (5), 716.4 (8), 734.4 (6), 816.4 (<5), 943.6 (25).

### 2.2.4. XRB-C976 (7)

Empirical formula: C₅₄H₈₀N₄O₁₂

Accurate mass: 976.5773

[M+H]⁺ (m/z): found: 977.5874; calculated: 977.5851

H/D exchange experiment: [M+D]⁺ m/z 977.59, [M+Na]⁺ m/z 999.57 (Rₜ=45.3 min)

ESI-MS/MS of m/z 977.6 (Rₜ=64.5 min, collision energy 25 and 85 V) [m/z (rel. int.)]: 100.1 (50), 126.1 (12), 134.1 (58), 168.1 (10), 200.1 (15), 210.1 (100), 228.1 (60), 244.1 (68), 262.1 (50), 328.2 (10), 362.2 (10), 455.3 (5), 489.3 (30), 507.3 (10), 589.4 (8), 716.4 (8), 750.4 (9), 850.4 (5), 977.6 (40).

### 2.2.5. XRB-C1011 (8)

Empirical formula: C₅₇H₇₈N₄O₁₂

Accurate mass: 1010.5616

[M+H]⁺ (m/z): found: 1011.5710; calculated: 1011.5695

H/D exchange experiment: [M+Na]⁺ m/z 1033.58 (Rₜ=43.3 min)

ESI-MS/MS of m/z 1011.6 (Rₜ=61.8 min, collision energy: 25 and 85 V) [m/z (rel. int.)]: 100.1 (36), 134.1 (100), 168.1 (5), 200.1 (8), 210.1 (58), 228.1 (36), 244.1 (100), 262.1 (78), 328.2 (5), 362.2 (10), 489.3 (18), 507.3 (5), 523.3 (15), 589.4 (5), 623.4 (5), 668.4 (6), 722.4 (5), 750.4 (10), 768.4 (5), 1011.6 (18).

### 2.2.6. XRB-C1044 (9)

Empirical formula: C₆₀H₇₆N₄O₁₂

Accurate mass: 1044.5460

[M+H]⁺ (m/z): found: 1045.5552; calculated: 1045.5538

H/D exchange experiment: [M+Na]⁺ m/z 1067.536 (Rₜ=41.4 min)

ESI-MS/MS of m/z 1045.6 (Rₜ=59.4 min, collision energy: 25 and 85 V) [m/z (rel. int.)]: 134.1 (60), 216.1 (9), 234.1 (16), 244.1 (100), 262.1 (70), 362.2 (17), 523.3 (12), 541.3 (9), 623.4 (5), 702.4 (6), 756.5 (7), 784.5 (7), 802.5 (8), 884.5 (5), 1045.6 (15).

### 2.2.7. XRB-E922 (10)

Empirical formula: C₄₉H₈₆N₄O₁₂

Accurate mass: 922.6242

[M+H]⁺ (m/z): found: 923.6338; calculated: 923.6321

H/D exchange experiment: [M+D]⁺ m/z 924.64, [M+Na]⁺ m/z 945.61 (Rₜ=64.4 min)

ESI-MS/MS of m/z 923.6 (Rₜ=93.6 min, collision energy: 25, 45, 75 V) [m/z (rel. int.)]: 100.1 (62), 126.1 (12), 168.1 (5), 200.1 (10), 210.1 (100), 224.1 (33), 228.1 (50), 242.2 (19), 328.2 (5), 342.2 (<5), 455.3 (10), 469.3 (10), 487.3 (<5), 555.4 (<5), 569.4 (<5), 600.4 (<5), 614.4 (<5), 668.4 (<5), 682.4 (<5), 696.4 (7), 714.4 (6), 796.5 (5), 923.6 (23).

### 2.2.8. XRB-E956 (11)

Empirical formula: C₅₂H₈₄N₄O₁₂

Accurate mass: 956.6086

[M+H]⁺ (m/z): found: 957.6178; calculated: 957:6164

H/D exchange experiment: [M+Na]⁺ m/z 979.6 (Rₜ=61.5 min)

ESI-MS/MS of m/z 957.6 (Rₜ=88 min, collision energy: 25, 45, 75 V) [m/z (rel. int.)]: 100.1 (60), 126.1 (13), 134.1 (25), 168.1 (10), 200.1 (10), 210.1 (100), 224.1 (<5), 228.1 (50), 258.1 (19), 276.1 (12), 328.2 (7), 342.2 (<5), 455.3 (12), 473.3 (<5), 503.3 (7), 521.3 (<5), 569.4 (<5), 682.4 (<5), 730.4 (6), 748.4 (5), 830.4 (<5), 957.6 (23).

ESI-MS/MS of m/z 957.6 (Rₜ=89.4 min, collision energy: 25, 45, 75 V) [m/z (rel. int.)]: 100.1 (85), 126.1 (13), 134.1 (35), 168.1 (8), 200.1 (11), 210.1 (100), 224.1 (45), 228.1 (60), 242.1 (25), 244.1 (28), 262.1 (21), 328.2 (<5), 342.2 (5), 455.3 (10), 469.3 (10), 489.3 (8), 503.3 (<5), 521.3 (<5), 569.4 (<5), 682.4 (<5), 714.4 (5), 730.4 (7), 748.4 (5), 830.4 (5), 957.6 (23).

ESI-MS/MS of m/z 957.6 (Rₜ=98.1 min, collision energy: 25, 45, 75 V) [m/z (rel. int.)]: 100.1 (55), 126.1 (33), 134.1 (40), 168.1 (8), 200.1 (11), 210.1 (100), 224.1 (25), 228.1 (51), 242.1 (25), 244.1 (35), 262.1 (28), 328.2 (8), 342.2 (5), 362.2 (<5), 455.3 (8), 469.3 (12), 489.3 (10), 503.3 (<5), 521.3 (<5), 569.4 (<5), 648.4 (<5), 696.4 (<5), 716.4 (8), 730.4 (7), 748.4 (5), 830.4 (8), 957.6 (33).

### 2.2.9. XRB-E990 (12)

Empirical formula: C₅₅H₈₂N₄O₁₂

Accurate mass: 990.5929

[M+H]⁺ (m/z): found: 991.6018; calculated: 991.6008

H/D exchange experiment: [M+Na]⁺ m/z 1013.58 (Rₜ=57.9 min)

ESI-MS/MS of m/z 991.6 (Rₜ=85.6 min, collision energy: 25 and 85 V) [m/z (rel. int.)]: 100.1 (88), 126.1 (15), 134.1 (100), 168.1 (8), 200.1 (19); 210.1 (92), 224.1 (65), 228.1 (55), 242.1 (32), 244.1 (78), 258.1 (18), 262.1 (58), 276.1 (15), 328.2 (5), 342.2 (5), 362.2 (8), 376.2 (5), 489.3 (21), 503.3 (22), 521.3 (8), 603.4 (8), 730.4 (9), 764.4 (8), 782.4 (6), 864.4 (6), 991.6 (60).

ESI-MS/MS of m/z 991.6 (Rₜ=93.4 min, collision energy: 25 and 85 V) [m/z (rel. int.)]: 100.1 (32), 126.1 (45), 134.1 (100), 168.1 (8), 200.1 (8), 210.1 (60), 224.1 (55), 228.1 (35), 242.1 (45), 244.1 (70), 262.1 (58), 342.2 (6), 362.2 (9), 489.3 (22), 503.3 (18), 589.4 (8), 648.4 (8), 730.4 (9), 764.4 (8), 782.4 (6), 864.4 (6), 991.6 (55).

### 2.2.10. XRB-E1024 (13)

Empirical formula: C₅₈H₈₀N₄O₁₂

Accurate mass: 1024.5773 [M+H]⁺ (m/z): found: 1025.5865; calculated: 1025.5851

ESI-MS/MS of m/z 1025.6 (Rₜ=81.6 min, collision energy: 25 and 85 V) [m/z (rel. int.)]: 100.1 (30), 134.1 (100), 210.1 (35), 224.1 (20), 228.1 (20), 234.1 (10), 242.1 (25), 244.1 (80), 258.1 (21), 262.1 (55), 276.1 (15), 362.2 (5), 376.2 (5), 489.3 (9), 503.3 (8), 523.3 (10), 537.3 (8), 603.4 (<5), 764.4 (15), 782.4 (6), 816.4 (5), 864.4 (6), 898.5 (6), 1025.6 (48).

### 2.2.11. XRB-S958 (14)

Empirical formula: C₄₇F₈₂N₄O₁₄S

Accurate mass: 958.5548

[M+H]⁺ (m/z): found: 959.5628; calculated: 959.5627 (FT-ICR-MS)

H/D exchange experiment: [M+Na]⁺ m/z 981.54 (Rₜ=16.0 min)

ESI-MS/MS of m/z 959.6 (Rₜ=21.2 min, collision energy: 25, 45, 75 V) [m/z (rel. int.)]: 70.1 (5), 100.1 (35), 126.1 (5), 150.1 (8), 168.1 (8), 180.1 (25), 200.1 (18), 210.1 (100), 228.1 (53), 260.1 (11), 278.1 (14), 328.2 (9), 378.2 (5), 455.3 (5), 505.3 (7), 523.3 (5), 555.4 (<5), 605.4 (<5), 732.4 (<5), 750.4 (<5), 832.5 (<5), 959.6 (12).

ESI-pseudo-MS³ of m/z 150.1 [m/z (rel. int.)]: 54 (4), 55 (8), 68 (2), 70.1 (69), 71.1 (17), 80.1 (2), 121.1 (2).

### 2.2.12. XRB-S992 (15)

Empirical formula: C₅₀H₈₀N₄O₁₄S

Accurate mass: 992.5392

[M+H]⁺ (m/z): found: 993.5480; calculated: 993.5470

H/D exchange experiment: [M+Na]⁺ m/z 1015.53 (Rₜ=14.9 min)

ESI-MS/MS of m/z 993.5 (Rₜ=19.9 min, collision energy. 25, 45, 75 V) [m/z (rel. int.)]: 70.1 (8), 100.1 (40), 126.1 (5), 134.1 (18), 150.1 (15), 168.1 (8), 180.1 (31), 200.1 (20), 210.1 (100), 228.1 (62), 244.1 (36), 260.1 (18), 262.1 (19), 278.1 (25), 328.2 (12), 360.3 (5), 455.3 (8), 505.3 (9), 539.3 (8), 605.4 (<5), 650.4 (5), 732.4 (<5), 766.4 (<5), 866.5 (<5), 993.6 (30).

ESI-MS/MS of m/z 993.5 (Rₜ=21.4 min, collision energy: 25, 45, 75 V) [m/z (rel. int.)]: 70.1 (5), 100.1 (25), 126.1 (8), 134.1 (25), 150.1 (12), 168.1 (5), 180.1 (35), 200.1 (18), 210.1 (100), 228 (48), 244.1 (19), 260.1 (15), 262.1 (22), 278.1 (21), 328.2 (8), 360.3 (5), 378.2 (8), 455.3 (5), 505.3 (9), 784.4 (5), 866.5 (5), 993.6 (18).

### 2.2.13. XRB-S1026 (16)

Empirical formula: C₅₃H₇₈N₄O₁₄S

Accurate mass: 1026.5235

[M+H]⁺ (m/z): found: 1027.5321; calculated: 1027.5314

H/D exchange experiment: [M+Na]⁺ m/z 1049.51 (Rₜ=14.0 min)

ESI-MS/MS of m/z 1027.5 (Rₜ=18.7 min, collision energy: 25, 45, 75 V) [m/z (rel. int.)]: 70.1 (7), 100.1 (30), 117.1 (5), 134.1 (35), 150.1 (10), 168.1 (5), 180.1 (30), 200.1 (15), 210.1 (40), 228 (40), 234.1 (20), 244.1 (100), 260.1 (18), 262.1 (41), 278.1 (24), 328.2 (10), 362.3 (12), 378.2 (9), 523.3 (11), 539 (8), 639.4 (6), 684.4 (5), 784.4 (5), 900.5 (8), 1027.6 (28).

ESI-MS/MS of m/z 1027.5 (Rₜ=20 min, collision energy: 25, 45, 75 V) [m/z (rel. int.)]: 70.1 (10), 100.1 (22), 134.1 (65), 150.1 (12), 168.1 (6), 180.1 (45), 200.1 (13), 210.1 (78), 228 (48), 234.1 (19), 244.1 (100), 260.1 (15), 262.1 (68), 278.1 (38), 328.2 (5), 362.2 (15), 378.2 (9), 439.3 (5), 457.3 (5), 489.3 (11), 505.3 (5), 523.3 (5), 539.3 (15), 639.4 (5), 684.4 (5), 750.4 (8), 768.4 (5), 784.4 (<5), 800.4 (5), 866.4 (5), 900.5 (8), 1027.6 (28).

### 2.2.14. XRB-S1060 (17)

Empirical formula: C₅₆F₇₆N₄O₁₄S

Accurate mass: 1060.5079

[M+H]⁺ (m/z): found: 1061.5165; calculated: 1061.5157

H/D exchange experiment: [M+Na]⁺ m/z 1083.50 (Rₜ=13.2 min)

ESI-MS/MS of m/z 1061.5 (Rₜ=18.9 min, collision energy: 25, 45, 85 V) [m/z (rel. int.)]: 134.1 (58), 150.1 (13), 180.1 (27), 234.1 (35), 244.1 (100), 260.1 (15), 262.1 (78), 278.1 (13), 362.2 (15), 523.3 (9), 539.3 (18), 557.3 (15), 639.4 (9), 772.4 (8), 818.4 (8), 1061.6 (22).

1. Previously unknown 24-membered cyclooctadepsipeptides (PF1022-V, PF1022-W, XRB-C894, XRB-C942, XRB-C976, XRB-C1010, XRB-C1044, XRB-E922, XRB-E956, XRB-E990, XRB-E1024, XRB-S958, XRB-S992, XRB-S1026, XRB-S1060), including their isomers.

2. Process for the preparation of the cyclooctadepsipeptides according to claim 1
a.) by using and culturing fungal strains from the family Xylariaceae, in particular from the genera Rosellinia and Coniolariella, and mitosporic fungal strains from the groups Fusarium, Beauveria and Verticillium (orders Hypocreales and Phyllachorales),
b.) by using the enzymatic preparations isolated from these fungal strains,
c.) and by subsequently isolating the cyclooctadepsipeptides from the culture.

3. Use of the cyclooctadepsipeptides according to claim 1 and of bassianolide individually or as a mixture as anthelmintics or endoparasiticides.
